(19) United States
(12) Patent Application Publication
(10) Pub. No.: US 2026/0199032 A1
(43) Pub. Date: Jul. 16, 2026

(54) SURGICAL ROBOTIC SYSTEMS, ADAPTER ASSEMBLIES AND SURGICAL LOADING UNITS THEREOF

(71) Applicant: COVIDIEN LP, Mansfield, MA (US)

(72) Inventors: Ronald P. LaRose, Cheshire, CT (US); Christopher P. Penna, Guilford, CT (US); David N. Fowler, Cheshire, CT (US); Jennifer C. Fremd, North Haven, CT (US); Rutuj Y. Shah, North Haven, CT (US); Jack R. Woods, Wallingford, CT (US); Brock Kopp, Colorado Springs, CO (US); Thomas W. Lennon, Marshfield, MA (US); Maria J. Rodriguez, Somerville, MA (US)

(21) Appl. No.: 19/136,367

(22) PCT Filed: Dec. 15, 2023
(86) PCT No.: PCT/IB2023/062790

§ 371 Date: Jun. 6, 2025

Related U.S. Application Data
(60) Provisional application No. 63/433,034, filed on Dec. 16, 2022.

(51) Int. Cl. A61B 34/30; A61B 17/00

(52) U.S. Cl. CPC A61B 34/30; A61B 17/00; A61B 2017/00398; A61B 2017/00486

(57) ABSTRACT

An adapter assembly of a surgical robotic system includes an elongate body configured to receive a surgical loading unit and an elongate loading bar coupled to the elongate body and configured to selectively lock the surgical loading unit to the adapter assembly. The elongate loading bar has a distal end defining a slot therein configured for receipt of an articulation link of the surgical loading unit upon an improper insertion of the surgical loading unit into the adapter assembly. The surgical robotic system is configured to automatically move the elongate loading bar between loading and unloading positions to allow for one-handed loading/unloading of the surgical loading unit.

This application claims the benefit of U.S. Provisional Patent Application No. 63/433,034, filed Dec. 16, 2022, the entire content of which is incorporated herein by reference.

### FIELD

The present technology is generally related to adapter assemblies for use with a surgical robotic system and methods of attaching and detaching a surgical loading unit to and from the adapter assembly.

### BACKGROUND

Surgical robotic systems are used in minimally invasive medical procedures because of their increased accuracy and expediency relative to handheld surgical instruments. In these surgical robotic systems, a robotic arm may support an instrument drive unit, which drives the operation of a surgical instrument. The surgical instrument may include an adapter assembly operably coupled to the adapter assembly, and a surgical loading unit that is detachably coupled to the adapter assembly. In operation, the robotic arm is moved to a position over a patient and then guides the surgical loading unit into a small incision via a surgical port or a natural orifice of a patient to position an end effector of the surgical loading unit at a work site within the patient's body.

### SUMMARY

The techniques of this disclosure generally relate to surgical robotic systems including adapter assemblies for interconnecting an instrument drive unit and a surgical loading unit. The adapter assemblies allow for one-handed unloading of a used surgical loading unit from the adapter assembly while the adapter assembly is coupled to the instrument drive unit. The disclosure also relates to mechanical features that ensure a proper assembly of the surgical loading unit with the adapter assembly whether the adapter assembly is connected to or disconnected from the instrument drive unit.

According to an aspect of the disclosure, a surgical robotic system is provided that includes a surgical loading unit, an adapter assembly, a processor, and a memory in communication with the processor. The adapter assembly is configured to be operably coupled to a surgical robotic arm and includes an elongate body and an elongate loading bar coupled to the elongate body and configured to move relative to the elongate body between a proximal position and a distal position. The elongate body has a distal end portion configured to couple to a proximal end portion of the surgical loading unit. In the proximal position, the elongate loading bar is configured to allow the surgical loading unit to be removable from the elongate body. In the distal position, the elongate loading bar is configured to secure the surgical loading unit to the elongate body. The processor is configured to execute the instructions to cause the system to automatically drive a proximal movement of the elongate loading bar to the proximal position in response to a first trigger threshold.

In aspects, the first trigger threshold may include the adapter assembly being moved proximally to a proximal position on the surgical robotic arm. The adapter assembly being moved proximally to the proximal position signifies a desire to remove the surgical loading unit from the adapter assembly.

In aspects, the processor may be further configured to automatically cause the system to drive a distal movement of the elongate loading bar to the distal position in response to a second trigger threshold. The second trigger threshold may include the surgical loading unit being removed from the elongate body.

In aspects, the adapter assembly may further include a drive screw configured to be rotated by a motor of the surgical robotic system, and a drive nut threadedly engaged to the drive screw and coupled to the elongate loading bar. Rotation of the drive screw may translate the elongate loading bar between the proximal and distal positions via the drive nut.

In aspects, the adapter assembly may further include a switch coupling the drive nut to the elongate loading bar such that movement of the drive nut along the drive screw is configured to move the elongate loading bar from the distal position to the proximal position via the switch.

In aspects, the surgical robotic system may further include a biasing member resiliently biasing the switch from a proximal position toward a distal position. When the switch is in the proximal position, the elongate loading bar may also be in the proximal position, and when the switch is in the distal position, the elongate loading bar may also be in the distal position.

In aspects, the processor may be further configured to automatically cause the system to drive a distal movement of the drive nut to move the switch to the distal position thereof in response to a second trigger threshold. The second trigger threshold may include the surgical loading unit being removed from the elongate body.

In aspects, the drive nut may include a flange received in an elongate slot defined in the switch. The processor, after causing the system to distally move the drive nut to distally move the switch to the distal position, may be further configured to automatically cause the system to drive a proximal movement of the flange of the drive nut within the elongate slot of the switch to position the flange at a proximal limit of the elongate slot. With the flange of the drive nut at the proximal limit of the elongate slot of the switch, a clinician is able to manually move the switch from the distal position to the proximal position without meeting resistance from the drive nut.

In aspects, the drive screw may have a multiple start thread (e.g., 5 threads) to allow for a resilient bias imparted on the drive nut by the biasing member to distally translate the drive nut along the drive screw whereby the drive nut rotates the drive screw.

In aspects, the elongate loading bar may have a distal end defining a slot therein configured for receipt of a proximal end portion of a component of the surgical loading unit upon an improper insertion of the surgical loading unit into the adapter assembly. The engagement of the proximal end portion of the component with the slot of the elongate loading bar resists rotation of the surgical loading unit relative to the adapter assembly toward an assembled state.

In accordance with another aspect of the disclosure, a surgical robotic system is provided that includes an instrument drive unit having a motor, an adapter assembly, a processor, and a memory in communication with the processor and having instructions stored therein. The adapter assembly includes a housing configured to be operably coupled to the instrument drive unit, a manual switch slidably coupled to the housing and operably to the motor of the instrument drive unit, an elongate body, and an elongate loading bar slidably coupled to the elongate body. The elongate body has a proximal end portion coupled to the housing, and a distal end portion configured to couple to a proximal end portion of a surgical loading unit. The elongate loading bar is coupled to the manual switch such that the elongate loading bar is configured to move relative to the elongate body from a distal position to a proximal position in response to proximal movement of the manual switch. The elongate loading bar is resiliently biased toward the distal position. The processor is configured to execute the instructions to cause the system to: actuate the motor of the instrument drive unit to drive a proximal movement of the elongate loading bar to the proximal position in response to a first trigger threshold signifying that the surgical loading unit is to be detached from the adapter assembly; and drive a distal movement of the elongate loading bar to the distal position in response to a second trigger threshold signifying that the surgical loading unit is detached from the adapter assembly.

In aspects, the surgical loading unit may be configured to be rotated into a locking engagement with the adapter assembly. The elongate loading bar may have a distal end defining a slot therein configured for receipt of a proximal end portion of a component of the surgical loading unit upon an improper insertion of the surgical loading unit into the adapter assembly. The engagement of the proximal end portion of the component with the slot of the elongate loading bar may resist rotation of the surgical loading unit relative to the adapter assembly toward the locking engagement with the adapter assembly.

In aspects, the adapter assembly may include a drive screw operably coupled to the motor of the instrument drive unit, and a drive nut threadedly engaged to the drive screw and coupled to the elongate loading bar via the manual switch such that rotation of the drive screw translates the elongate loading bar between the proximal and distal positions.

In aspects, the drive nut may include a flange received in an elongate slot defined in the manual switch. The flange of the drive nut may be configured to move between a proximal limit of the elongate slot and a distal limit of the elongate slot without moving the manual switch. The processor, after causing the system to move the elongate loading bar to the distal position, may be further configured to cause the system to drive a proximal movement of the flange of the drive nut within the elongate slot of the switch to position the flange at the proximal limit of the elongate slot.

In aspects, the adapter assembly may further include a biasing member resiliently biasing the elongate loading bar to the distal position. The drive screw may have a multiple start thread to allow for rotation of the drive screw during distal translation of the drive nut along the drive screw due to the resilient bias imparted on the drive nut by the biasing member.

In accordance with another aspect of the disclosure, a method of exchanging a surgical loading unit in a surgical robotic system is provided. The method includes determining that a surgical loading unit is to be detached from an adapter assembly of the surgical robotic system; and upon the system determining that the surgical loading unit is to be detached from the adapter assembly, actuating a motor of an instrument drive unit of the surgical robotic system to drive a proximal movement of an elongate loading bar of the adapter assembly to a proximal position, whereby the elongate loading bar unlocks the surgical loading unit from the adapter assembly.

In aspects, the method may further include actuating the motor of the instrument drive unit to drive a distal movement of the elongate loading bar to a distal position upon the system determining that the surgical loading unit is detached from the adapter assembly.

In aspects, the method may further include actuating the motor of the instrument drive unit to drive a proximal movement of a drive nut relative to a manual switch of the adapter assembly after the elongate loading bar is moved distally to the distal position.

In aspects, the method may further include distally moving, via a biasing member of the adapter assembly, the elongate loading bar to the distal position upon detachment of the adapter assembly from the instrument drive unit.

In aspects, the biasing member may move the elongate loading bar distally against a resistive axial force of a drive nut of the adapter assembly that intercouples a drive screw of the adapter assembly to the elongate loading bar.

In aspects, determining that the surgical loading unit is to be detached may include determining that the adapter assembly is moved proximally to a proximal position on a surgical robotic arm of the surgical robotic system.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Aspects of the present disclosure are described herein with reference to the accompanying drawings, wherein:

FIG. 1 is a perspective view, with parts separated, of components of a hand-held, electromechanical surgical instrument including a handle assembly, an adapter assembly, and a surgical loading unit;

FIG. 2 is a perspective view of a proximal end portion of the surgical loading unit of FIG. 1;

FIG. 3 is a front view of a ring member of the adapter assembly of FIG. 1 which receives the proximal end portion of the surgical loading unit;

FIG. 4 is a perspective view of the proximal end portion of the surgical loading unit being properly inserted into the ring member of FIG. 3;

FIG. 5 is a side, perspective view illustrating a first improper insertion of the surgical loading unit into the adapter assembly;

FIG. 6 is a side, perspective view illustrating a first side of the surgical instrument during a second improper insertion of the surgical loading unit into the adapter assembly;

FIG. 7 is a perspective view illustrating another side of the surgical instrument during the second improper insertion of the surgical loading unit into the adapter assembly;

FIG. 8 is a schematic illustration of a surgical robotic system including a control tower, a console, and one or more surgical robotic arms;

FIG. 9 is a perspective view illustrating a surgical assembly of the surgical robotic system of FIG. 8 including a slide of the surgical robotic arm, an instrument drive unit coupled to the slide, an adapter assembly coupled to the instrument drive unit, and a surgical loading unit coupled to the adapter assembly;

FIG. 10 is a side view, with parts removed to show inner components, illustrating the adapter assembly and the surgical loading unit of FIG. 9 in a coupling state;

FIG. 11 is a side view illustrating the surgical loading unit lockingly engaged with the adapter assembly;

FIG. 12 is a side view, with parts removed, illustrating internal components of the adapter assembly of FIG. 9;

FIG. 13 is a perspective view, with parts removed, illustrating the internal components of the adapter assembly of FIG. 12 isolated from a remainder of the adapter assembly;

FIG. 14 is a perspective view, with parts separated, of the internal components of the adapter assembly of FIG. 13;

FIG. 15A is an enlarged side, perspective view illustrating an actuation switch and a drive nut of the adapter assembly of FIG. 12 in an unloading position;

FIG. 15B is an enlarged side, perspective view illustrating the actuation switch and the drive nut in a locked position;

FIG. 15C is an enlarged side, perspective view illustrating the drive nut in a proximal position relative to the actuation switch;

FIG. 15D is an enlarged side, perspective view illustrating the actuation switch in a proximal, loading position;

FIG. 16 is a flow chart illustrating a method of performing a surgical loading unit exchange with the adapter assembly;

FIG. 17A is a front perspective view of the actuation switch of FIG. 15A;

FIG. 17B is a front view of the actuation switch;

FIG. 17C is a rear view of the actuation switch;

FIG. 17D is a side view of the actuation switch;

FIG. 17E is a top view of the actuation switch; and

FIG. 17F is a bottom view of the actuation switch.

### DETAILED DESCRIPTION

As used herein, the terms parallel and perpendicular are understood to include relative configurations that are substantially parallel and substantially perpendicular up to about + or −10 degrees from true parallel and true perpendicular.

Aspects of the presently disclosed surgical systems are described in detail with reference to the drawings, in which like reference numerals designate identical or corresponding elements in each of the several views. As used herein, the term “distal” refers to that portion of the surgical system closer to a surgical site, while the term “proximal” refers to that portion of the surgical system farther from the surgical site.

Presently, if a surgical loading unit is inserted incorrectly into an adapter assembly and rotated after the incorrect insertion, an annular member or rotating ring of the adapter assembly is caused to be rotated out of a normal position. After the surgical loading unit is removed, the rotating ring remains out of the normal position. As such, a subsequent attempt at inserting a surgical loading unit into the adapter assembly is prohibited due to the rotating ring being displaced from its normal operating position.

The disclosure provides a surgical instrument that includes a surgical loading unit and an adapter assembly that interconnects the surgical loading unit with either a handle assembly or a robotic assembly. The adapter assembly includes a plurality of mechanical features that ensure that the surgical loading unit is connected to the adapter assembly in a proper orientation to prevent the improper displacement of the rotating ring. Further, the adapter assembly is configured to allow for a one-handed loading and unloading of the surgical loading unit to and from the adapter assembly while the adapter assembly is attached to a surgical robotic arm.

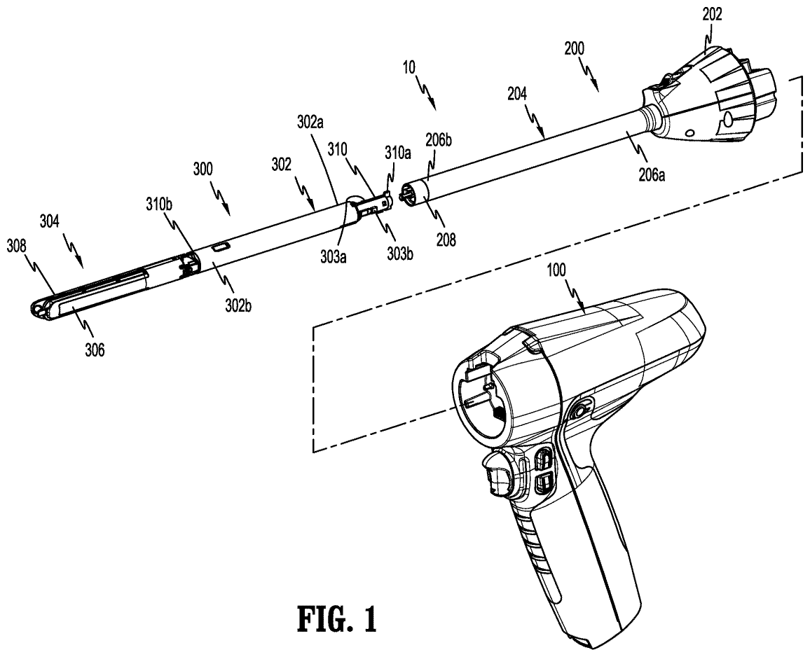

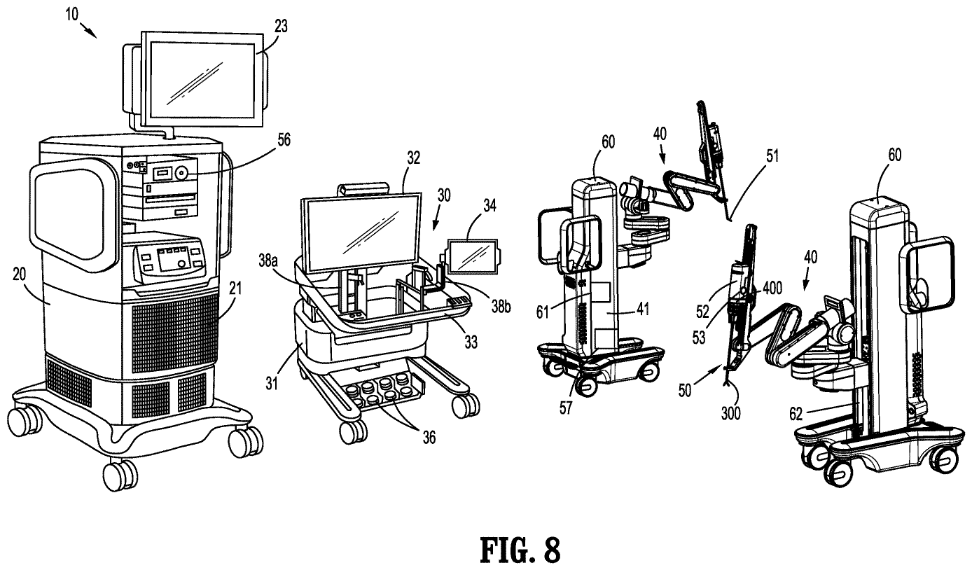

With reference to FIG. 1, a surgical instrument 10, in accordance with an aspect of the disclosure, is shown as a powered, hand-held, electromechanical surgical instrument. The surgical instrument 10 includes a handle assembly 100 configured for selective connection with any one of a number of adapter assemblies 200, and, in turn, each unique adapter assembly 200 is configured for selective connection with any number of surgical loading units 300. The surgical loading unit 300 and adapter assembly 200 are configured for actuation and manipulation by the handle assembly 100 or, in aspects, a surgical robotic system 10, as will be described with reference to FIG. 8.

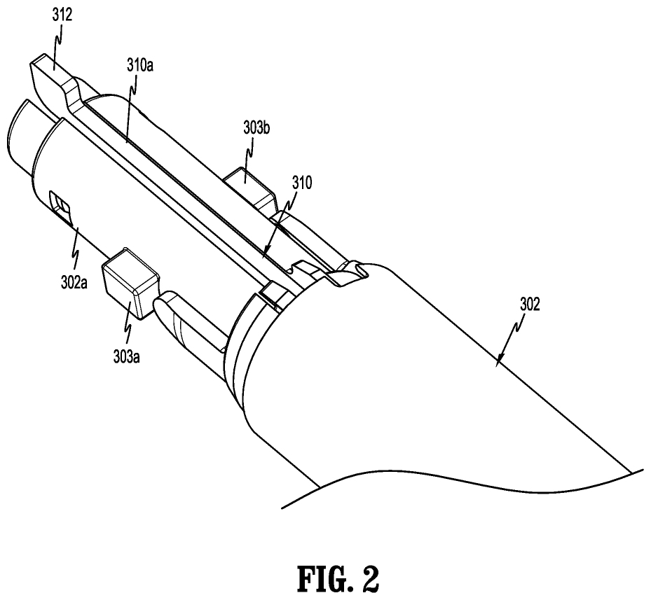

With reference to FIGS. 1 and 2, the surgical loading unit 300 of the surgical instrument 10 has a proximal body portion 302 and a tool assembly or end effector 304 coupled to a distal end portion 302b of the proximal body portion 302. The proximal body portion 302 has a proximal end portion 302a configured for engagement with a distal end portion 206b of an elongate body 204 of the adapter assembly 200. The proximal body portion 302 has a pair of surface features, such as, for example, lugs 303a, 303b extending outwardly from opposite sides of the proximal end portion 302a of the surgical loading unit 300. The lugs 303a, 303b may assume any suitable shape, such as a square or a cylinder. The end effector 304 is pivotally attached to the proximal body portion 302 and includes an anvil assembly 306 and a cartridge assembly 308. The cartridge assembly 308 is pivotable in relation to the anvil assembly 306 and is movable between an open or unclamped position and a closed or clamped position for insertion through a cannula of a trocar. In aspects, the end effector 304 may be configured to perform alternate functions, such as, electrosurgical sealing.

The surgical loading unit 300 further includes an articulation link 310 extending through the proximal body portion 302 and centrally between the lugs 303a, 303b. The articulation link 310 has a proximal end portion 310a having a flag 312 protruding proximally and radially outward from the proximal body portion 302. The flag 312 of the articulation link 310 is configured to operably couple to an articulation drive member (not explicitly shown) of the adapter assembly 200 for driving a translation of the articulation link 310. The articulation link 310 has a distal end portion 310b operably coupled to the end effector 304, such that the end effector 304 is configured to articulate relative to the proximal body portion 302 in response to a translation of the articulation link 310. For example, the end effector 304 is movable from a first position in which the end effector 304 is aligned with a longitudinal axis of the proximal body portion 302 to at least a second position in which the end effector 304 is disposed at a non-zero angle with respect to the longitudinal axis of the proximal body portion 302.

With further reference to FIG. 1, the adapter assembly 200 includes a knob housing 202 and an elongate body 204 extending from a distal end of the knob housing 202. The knob housing 202 and elongate body 204 are configured and dimensioned to house the components of the adapter assembly 200. The elongate body 204 may be dimensioned for endoscopic insertion. In aspects, the elongate body 204 may be passable through a typical trocar port, cannula or the like. The knob housing 202 may be dimensioned to not enter the trocar port, cannula or the like. The elongate body 204 has a proximal end portion 206a attached to the knob housing 202, which is configured to be attached to the handle assembly 100. The elongate body 204 also includes a distal end portion 206b configured to be coupled to the proximal body portion 302 of the surgical loading unit 300.

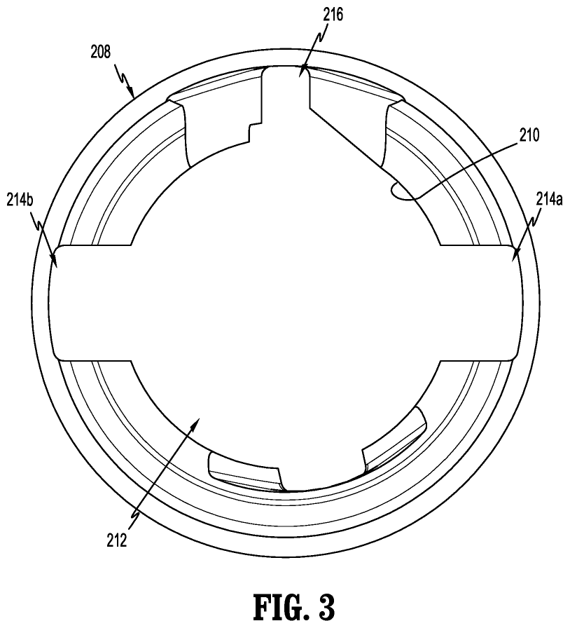

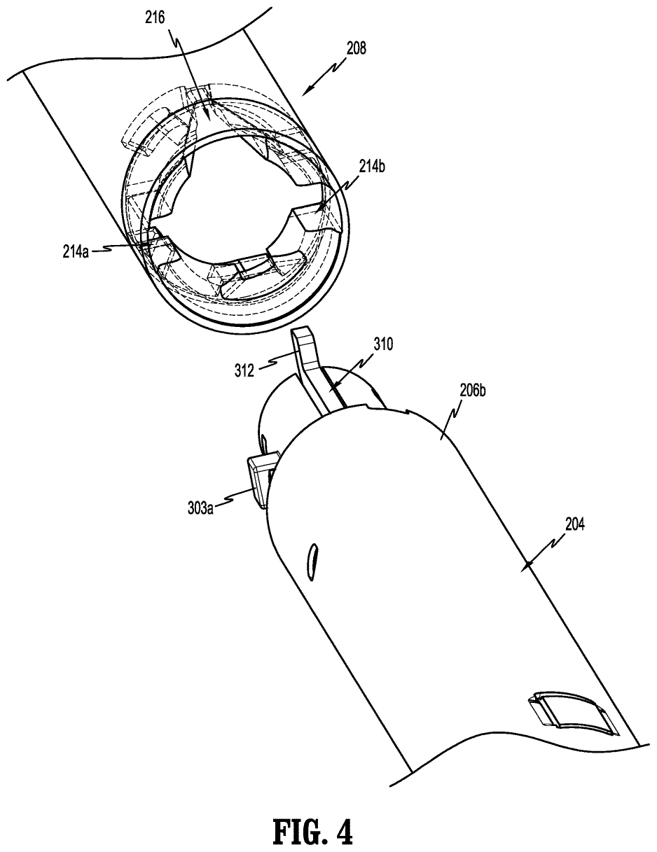

With reference to FIGS. 1, 3, and 4, the elongate body 204 of the adapter assembly 200 further includes a distal cap or ring member 208 extending distally from the distal end portion 206b. In aspects, the ring member 208 may be formed with the elongate body 204 and/or may be housed therein. The ring member 208 has an inner surface 210 that defines an opening or channel 212 configured for receipt of the proximal end portion 302a of the proximal body portion 302 of the surgical loading unit 300. The inner surface 210 of the ring member 208 further defines a pair of diametrically opposed apertures 214a, 214b and a slot 216 each being circumferentially disposed about the ring member 208. The slot 216 is disposed between the apertures 214a, 214b and is spaced circumferentially from each of the apertures 214a, 214b by about 90 degrees. The apertures 214a, 214b are configured for receipt of the respective pair of lugs 303a, 303b of the surgical loading unit 300 and the slot 216 is configured for receipt of the proximal end portion 310a (e.g., the flag 312) of the articulation link 310 of the surgical loading unit 300 during a proper insertion of the surgical loading unit 300 into the adapter assembly 200, as shown in FIG. 4.

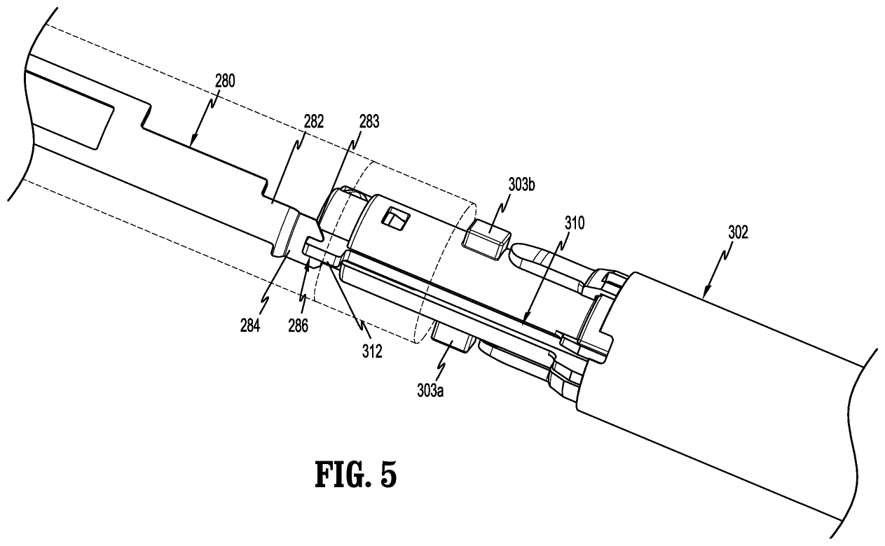

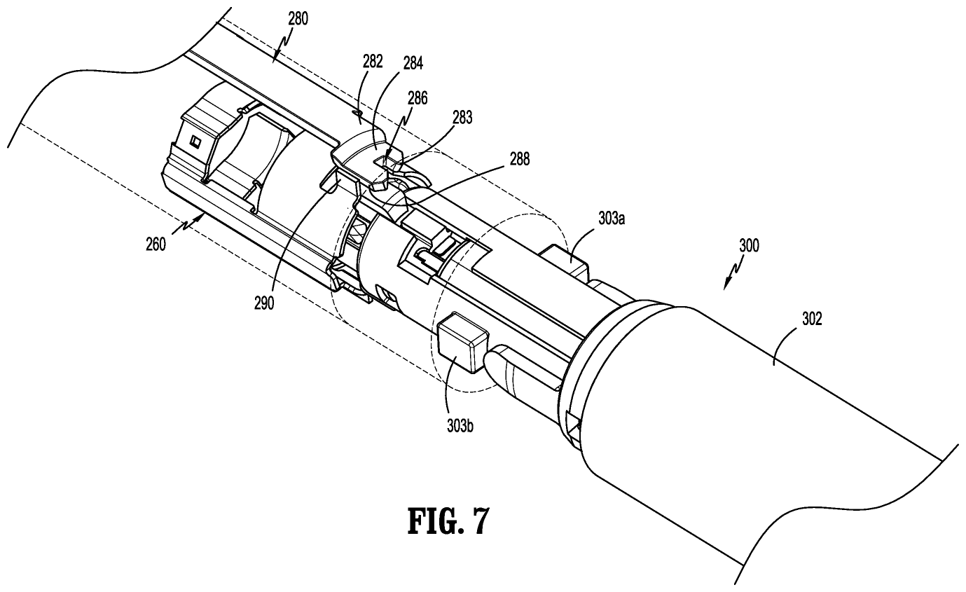

With reference to FIGS. 5 and 7, the adapter assembly 200 further includes an elongate loading bar or locking link 280 disposed within the elongate body 204 of the adapter assembly 200. The elongate loading bar 280 is slidingly disposed within the elongate body 204 and is resiliently biased toward a distal, locking position, as shown in FIG. 5. The elongate loading bar 280 has a distal extension 282 configured for locking engagement with the lug 303a (FIG. 2) of the surgical loading unit 300 upon the proper insertion of the surgical loading unit 300 into elongate body 204. The distal extension 282 has a distal end 284 having a distally-facing edge 283 defining a slot 286 therein. The slot 286 has a similar shape and size as the flag 312 of the surgical loading unit 300 to accommodate the flag 312 therein during an improper insertion of the surgical loading unit 300 into adapter assembly 200 (FIG. 5). The slot 286 as illustrated has a rectangular shape, but other suitable shapes are contemplated, such as rounded, triangular, or the like.

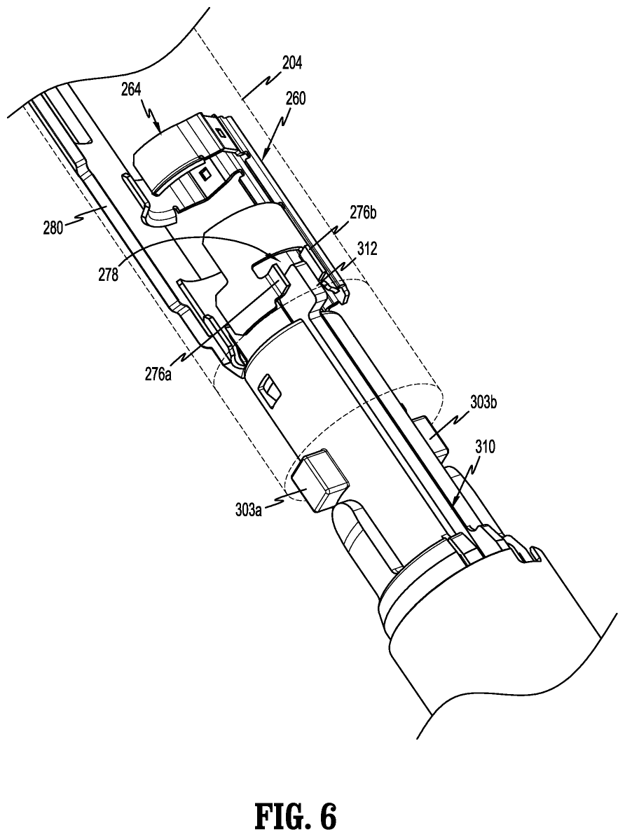

With reference to FIGS. 6 and 7, the adapter assembly 200 further includes an annular member 260 rotatably disposed within the elongate body 204 of the adapter assembly 200. The annular member 260 functions to electromechanically communicate to a processor (not shown) of the handle assembly 100 that the surgical loading unit 300 is either properly or improperly connected to the adapter assembly 200. In particular, upon rotating the annular member 260 relative to the elongate body 204, about a longitudinal axis of the elongate body 204, from a starting or first orientation to a second orientation, the annular member 260 transmits a signal to the processor of the handle assembly 100 indicating that the surgical loading unit 300 is secured to the adapter assembly 200 and is ready for use.

The annular member 260 defines a cylindrical passageway 264 therethrough configured for disposal of the proximal body portion 302 of the surgical loading unit 300. The annular member 260 includes a surface feature, such as, for example, a pair of tabs 276a, 276b defining a cavity 278 therebetween configured to interface with the lug 303b of the surgical loading unit 300, such that the annular member 260 is rotatable by and with the surgical loading unit 300 when the surgical loading unit 300 is properly inserted into the adapter assembly 200.

The annular member 260 further includes an appendage or additional surface feature 290 protruding radially outward therefrom and disposed on an opposite side of the annular member 260 as the pair of tabs 276a, 276b. The appendage or tab 290 is positioned in abutting engagement with a lateral edge surface 288 of the distal extension 282 of the elongate loading bar 280 (FIG. 7) when the elongate loading bar 280 is in the distal position. The elongate loading bar 280 prevents the annular member 260, and in turn, the surgical loading unit 300, from being rotated relative to the elongate body 204 due to the engagement of the appendage 290 of the annular member 260 with the elongate loading bar 280. As such, only when the lug 303a of the surgical loading unit 300 engages and proximally moves the elongate loading bar 280 out of engagement with the appendage 290 (during a proper insertion of the surgical loading unit 300) will the annular member 260 be able to be rotated by the surgical loading unit 300.

In operation, to properly assemble the surgical loading unit 300 with the adapter assembly 200, the surgical loading unit 300 is rotationally oriented (about a longitudinal axis thereof) so that the pair of lugs 303a, 303b of the surgical loading unit 300 are aligned with the pair of apertures 214a, 214b of the ring member 260 and the flag 312 of the articulation link 310 of the surgical loading unit 300 is aligned with the slot 216 of the ring member 208, as shown in FIG. 4. With the surgical loading unit 300 properly oriented, the surgical loading unit 300 may be translated toward the adapter assembly 200 to pass the proximal body portion 302 of the surgical loading unit 300 into the elongate body 204 of the adapter assembly 200 and, in turn, into the annular member 260. Upon fully inserting the surgical loading unit 300 into the adapter assembly 200, the lug 303b of the surgical loading unit 300 is received between the surface features 276a, 276b of the annular member 260, the lug 303a of the surgical loading unit 300 engages the elongate loading bar 280 to retract the elongate loading bar 280 towards its proximal position, and the flag 312 of the articulation link 310 couples to the articulation drive member (not shown) of the adapter assembly 200.

After moving the elongate loading bar 280 to the proximal position by the lug 303a of the surgical loading unit 300, the distal extension 282 of the elongate loading bar 280 is no longer engaged with the appendage 290 of the annular member 260, and therefore no longer preventing the annular member 260 from rotating out of the first orientation. With the surgical loading unit 300 in this initial insertion position within the adapter assembly 200, the surgical loading unit 300 is not yet lockingly engaged with the adapter assembly 200 and the annular member 260 remains in the first orientation. To complete the mechanical coupling of the surgical loading unit 300 to the adapter assembly 200, the surgical loading unit 300 is then rotated relative to the elongate body 204. Since the lug 303b of the surgical loading unit 300 is received in the cavity 278 defined between the surface features 276a, 276b of the annular member 260, rotation of the surgical loading unit 300 drives a rotation of the annular member 260 from the first orientation to the second orientation. Rotation of the annular member 260 from the first orientation to the second orientation establishes an electrical connection between the annular member 260 and the processor of the handle assembly 100, whereby the processor registers that the surgical loading unit 300 is lockingly engaged with the adapter assembly 200 and surgical instrument 10 is ready for operation.

The rotation of the surgical loading unit 300 moves the lug 303a of the surgical loading unit 300 into an inner groove (not explicitly shown) defined in the ring member 208 of the elongate body 204 and out of a longitudinal path of the elongate loading bar 280. The resilient bias of the elongate loading bar 280 drives an axial translation thereof to dispose the elongate loading bar 280 in the distal or locking position. With the elongate loading bar 280 in the distal position, the lug 303a of the surgical loading unit 300 is captured between the ring member 208 and the distal extension 282, thereby preventing the surgical loading unit 300 from sliding or rotating out of the adapter assembly 200. In this state, the surgical loading unit 300 is properly releasably, lockingly engaged to the adapter assembly 200 and ready for use.

In some instances, it is possible for a clinician to inadvertently improperly orient the surgical loading unit 300 (about a longitudinal axis thereof) relative to the adapter assembly 200 prior to inserting the surgical loading unit 300 into the adapter assembly 200. For example, with reference to FIG. 5, the surgical loading unit 300 may be improperly oriented 90 degrees counter-clockwise (about the longitudinal axis thereof) from the proper orientation. When the rotational orientation of the surgical loading unit 300 is improper, the surgical loading unit 300 may still be longitudinally inserted into the adapter assembly 200. However, in this orientation, instead of the lug 303a of the surgical loading unit 300 engaging the distal extension 282 of the elongate loading bar 280, the flag 312 of the articulation link 310 is received in the slot 286 of the distal extension 282. Accordingly, when the clinician attempts to complete the assembly of the surgical loading unit 300 with the adapter assembly 200 by exerting a rotational force on the surgical loading unit 300, the engagement of the flag 312 of the articulation link 310 with the slot 286 of the elongate loading bar 280, which is non-rotatable, advantageously prevents the surgical loading unit 300 from being rotated. Therefore, the clinician will be unable to operate the surgical instrument 10 and will be alerted to the fact that the surgical loading unit 300 is improperly oriented.

With reference to FIGS. 6 and 7, the surgical loading unit 300 may be improperly oriented 90 degrees clockwise (about the longitudinal axis thereof) from the proper orientation. In this orientation, the flag 312 of the articulation link 310 is received in the cavity 278 defined by the pair of surface features 276a, 276b of the annular member 260 instead of the lug 303b of the surgical loading unit 300, as shown in FIG. 6. In addition, neither lug 303a nor lug 303b of the surgical loading unit 300 will engage the distal extension 282 of the elongate loading bar 280, such that the distal extension 282 remains engaged with the appendage 290 of the annular member 260. Accordingly, when the clinician attempts to complete the assembly of the surgical loading unit 300 with the adapter assembly 200 by exerting a rotational force on the surgical loading unit 300, the engagement of the appendage 290 with the distal extension 282 advantageously prevents the annular member 260, and in turn, the surgical loading unit 300 from being rotated. Therefore, the clinician will be unable to operate the surgical instrument 10 and will be alerted to the fact that the surgical loading unit 300 is improperly oriented.

With reference to FIG. 8, a surgical robotic system 10 is provided that includes an adapter assembly 400 having similar mechanical features of the adapter assembly 200 described with reference to FIGS. 1-7 for ensuring that the surgical loading unit 300 is connected to the adapter assembly 400 in a proper orientation. The surgical robotic system 10 generally includes a control tower 20, which is connected to all of the components of the surgical robotic system 10 including a surgical console 30 and one or more robotic arms 40. Each of the robotic arms 40 includes a surgical instrument 50 and instrument drive unit 52 removably coupled thereto. Each of the robotic arms 40 is also coupled to and supported on a movable robotic arm cart 60.

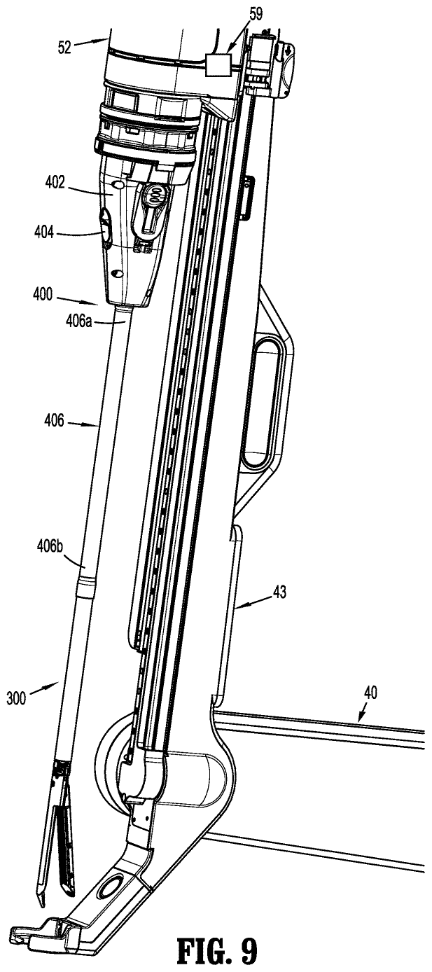

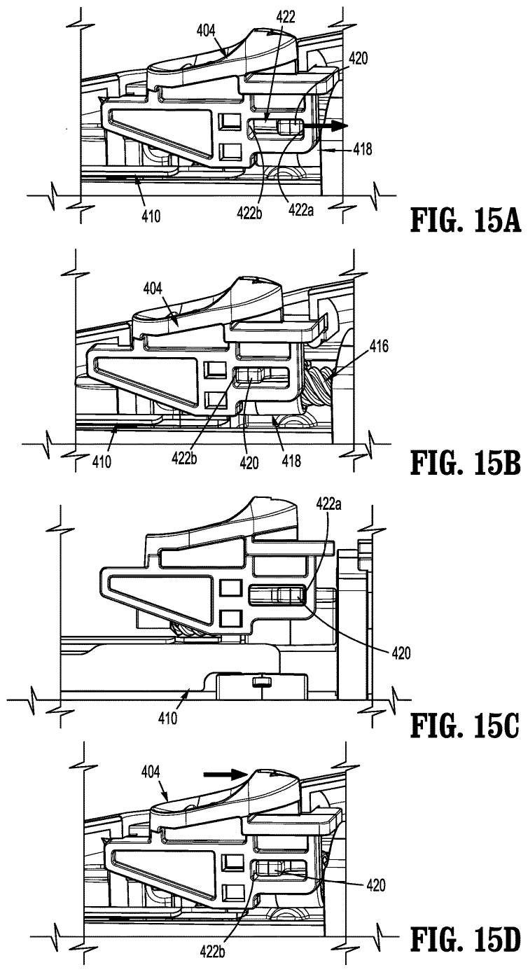

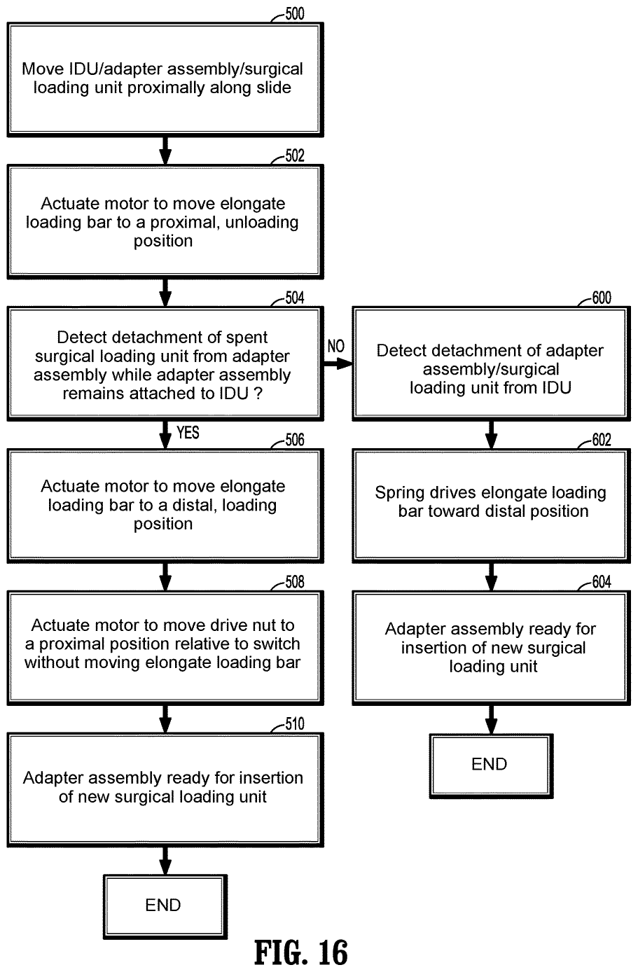

The surgical instrument 50 includes the adapter assembly 400 coupled to the instrument drive unit 52 and a surgical loading unit 300 detachably coupled to the adapter assembly 400, as will be further described with reference to FIGS. 9-14. The surgical loading unit 300 is configured for use during minimally invasive surgical procedures. In embodiments, the surgical loading unit 300 may be configured for open surgical procedures. In embodiments, the surgical loading unit 300 may be an endoscope, such as an endoscopic camera 51, configured to provide a video feed for the user. In further embodiments, the surgical loading unit 300 may be an electrosurgical forceps configured to seal tissue by compressing tissue between jaw members and applying electrosurgical current thereto. In yet further embodiments, the surgical loading unit 300 may be a surgical stapler.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the disclosure and output the processed video stream.

The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor 57 operably connected to a memory 61, which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor 57 may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor 57 may be substituted for by using any logic processor (e.g., control circuit) adapted to execute algorithms, calculations, and/or set of instructions described herein. The robotic arm 40 also includes a plurality of manual override buttons 53 disposed on the instrument drive unit 52 and the setup arm 62, which may be used in a manual mode. The user may press one or more of the buttons 53 to move the component associated with the button 53.

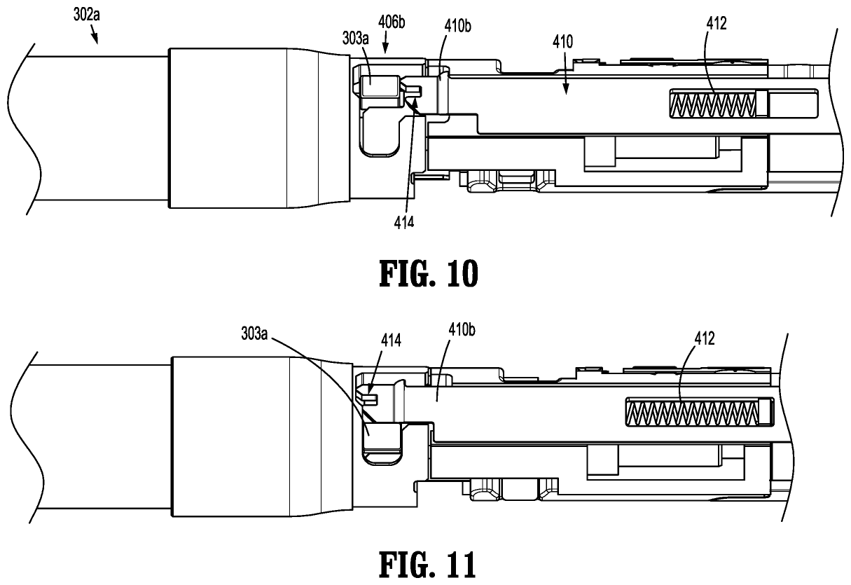

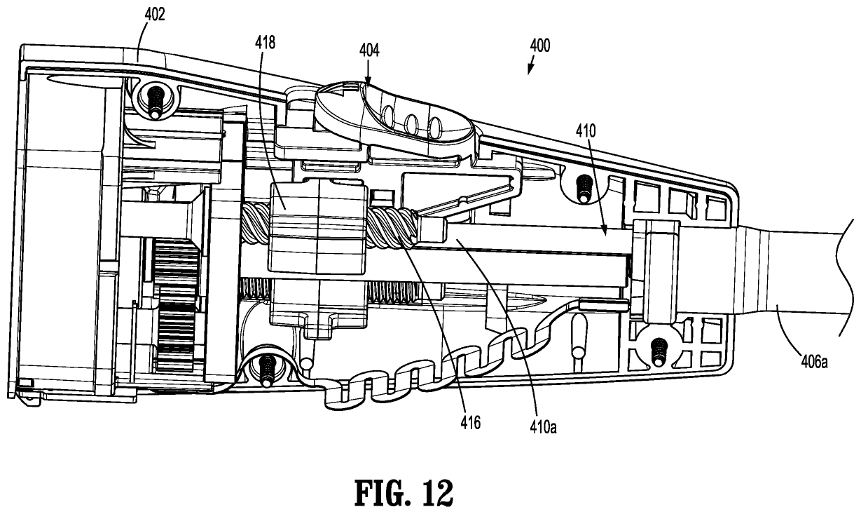

With reference to FIGS. 9-11, the instrument drive unit 52 is slidably coupled to a slide 43 of the surgical robotic arm 40 such that the instrument drive unit 52 is movable between a plurality of positions along a length of the slide 43. The adapter assembly 400 of the surgical robotic system 10 includes a housing 402 configured to be detachably coupled to the instrument drive unit 52, a manual switch 404 slidably coupled to the housing 402 and operably coupled to the motor 59 of the instrument drive unit 52, an elongate tube or body 406, and an elongate loading bar 410 (FIGS. 10-11) slidably supported in the elongate body 406. The elongate body 406 has a proximal end portion 406a coupled to the housing 402, and a distal end portion 406b configured to receive the proximal end portion 302a of the surgical loading unit 300.

With reference to FIGS. 10-14, the elongate loading bar 410 of the adapter assembly 400 has a proximal end portion 410a and a distal end portion 410b. The proximal end portion 410a of the elongate loading bar 410 is coupled to the manual switch 404 such that the elongate loading bar 410 is configured to move relative to the elongate body 406 from a distal position (FIG. 11) to a proximal position (FIG. 10) in response to proximal movement of the manual switch 404. The elongate loading bar 410 is resiliently biased toward the distal position by a biasing member, such as, for example, a spring 412 supported in the elongate body 406.

The distal end portion 410b of the elongate loading bar 410 is configured to be urged proximally by one of the lugs 303a or 303b extending outwardly from the proximal end portion 302a of the surgical loading unit 300 upon axial insertion of the proximal end portion 302a of the surgical loading unit 300 into the distal end portion 406b of the elongate body 406 of the adapter assembly 400, as shown in FIG. 10. When the surgical loading unit 300 is attached to the adapter assembly 400 and the elongate loading bar 410 is in the distal position, as shown in FIG. 11, the distal end portion 410b of the elongate loading bar 410 lockingly engages the surgical loading unit 300 with the adapter assembly 400 by preventing the lug 303a of the surgical loading unit 300 from rotating out of the locked state. On the other hand, when the surgical loading unit 300 is attached to the adapter assembly 400, and the elongate loading bar 410 is in the proximal position, as shown in FIG. 10, the surgical loading unit 300 may be rotated and then withdrawn from the adapter assembly 400.

The distal end portion 410b of the elongate loading bar 410 defines a slot 414 therein configured for receipt of a proximal end portion of a component of the surgical loading unit 300 (e.g., the proximal end portion 310a of the articulation link 310, FIG. 5) upon an improper insertion of the surgical loading unit 300 into the adapter assembly 400, whereby the engagement of the proximal end portion 310a of the articulation link 310 with the slot 414 of the elongate loading bar 410 resists rotation of the surgical loading unit 300 relative to the adapter assembly 400 toward the assembled state shown in FIG. 11. Further details about the elongate loading bar 410 and its mechanism for preventing an improper insertion of the surgical loading unit 300 are provided above with reference to FIGS. 1-7.

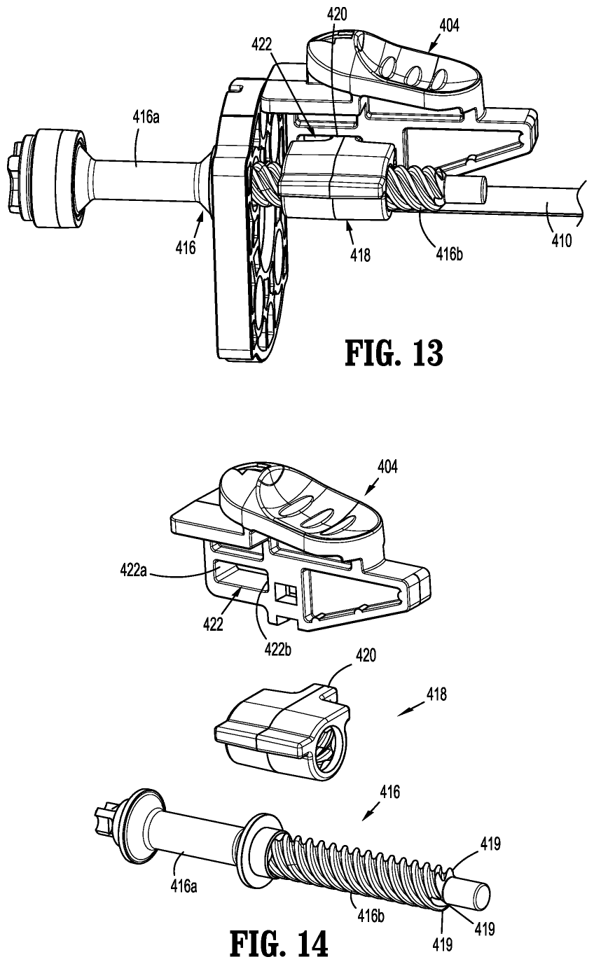

With reference to FIGS. 12-14, the switch 404 of the adapter assembly 400 is axially fixed to the proximal end portion 410a of the elongate loading bar 410 such that proximal or distal movement of the switch 404 causes a corresponding proximal or distal movement of the elongate loading bar 410 and proximal or distal movement of the elongate loading bar 410 causes a corresponding proximal or distal movement of the switch 404. The switch 404 is configured to move between a proximal position, in which the elongate loading bar 410 is in the proximal position, and a distal position, in which the elongate loading bar 410 is in the distal position. When the switch 404 is moved to the proximal position, the system 10 may provide a notification to the clinician (e.g., an audible alert, haptic feedback, a color change, a prompt on the display 23, 32, or 34, or a combination thereof) that the surgical loading unit 300 is in an unlocked state, and therefore unsafe for use. In aspects, when the switch 404 is moved to the proximal position, the system 10 may be configured to prohibit actuation of any motors of the instrument drive unit 52 that may drive an operation of the surgical loading unit 300. When the switch 404 is in the distal position, the system 10 may provide an audible, visible and/or haptic alert to the clinician that the surgical loading unit 300 is locked to the adapter assembly 400, and is therefore safe for use.

The adapter assembly 400 further includes a drive nut 418 operably coupled to a drive screw 416 each of which being supported in the housing 402 of the adapter assembly 400. The drive nut 416 is coupled to the elongate loading bar 410 via the manual switch 404 of the adapter assembly 400 such that movement of the drive nut 418 along the drive screw 416 is configured to move the elongate loading bar 410 from the distal position to the proximal position via the switch 404. More specifically, the drive nut 418 includes a laterally-extending appendage or flange 420 received in a longitudinally-extending elongate slot 422 defined in a body of the switch 404. The elongate slot 422 has a proximal limit 422a and a distal limit 422b between which the flange 420 of the drive nut 418 is configured to translate. As such, only when the flange 420 of the drive nut 418 is engaged to the proximal limit 422a of the elongate slot 422 will proximal movement of the drive nut 418 cause a corresponding proximal movement of the switch 404 and the attached elongate loading bar 410. Similarly, only when the flange 420 of the drive nut 418 is engaged to the distal limit 422b of the elongate slot 422 will distal movement of the drive nut 418 cause a corresponding distal movement of the switch 404 and the attached elongate loading bar 410.

The drive screw 416 has a proximal end portion 416a configured to be drivingly coupled to a drive shaft (not explicitly shown) of a drive motor 59 (FIG. 9) of the instrument drive unit 52. Actuation of the drive motor 59 rotates the drive screw 416 about its longitudinal axis and relative to the housing 402 of the adapter assembly 400. When the motor 59 of the instrument drive unit 52 is operably coupled to the elongate loading bar 410 via the switch 404, the drive nut 418, and the drive screw 416, manual movement of the elongate loading bar 410 via the manual switch 404 is resisted. On the other hand, when the adapter assembly 400 is decoupled from the instrument drive unit 52, manual operation of the elongate loading bar 410 via the switch 404 is permitted.

The drive screw 416 has a threaded distal end portion 416b threadedly coupled to the drive nut 418 such that rotation of the drive screw 416 is configured to translate the drive nut 418 along the drive screw 416. The threaded distal end portion 416b of the drive screw 416 may include a multiple start thread 419 (e.g., 5 threads) to allow for rotation of the drive screw 416 during distal translation of the drive nut 418 along the drive screw 416 due to a resilient bias imparted on the drive nut 418 by the spring 412. The spring constant of the spring 412 is selected to allow the spring 412 to overcome any resistance to translation of the drive nut 418 along the drive screw 416. Consequently, with the adapter assembly 400 decoupled from the instrument drive unit 52, the spring 412 is configured to automatically drive a distal translation of the elongate loading bar 410 to the distal position.

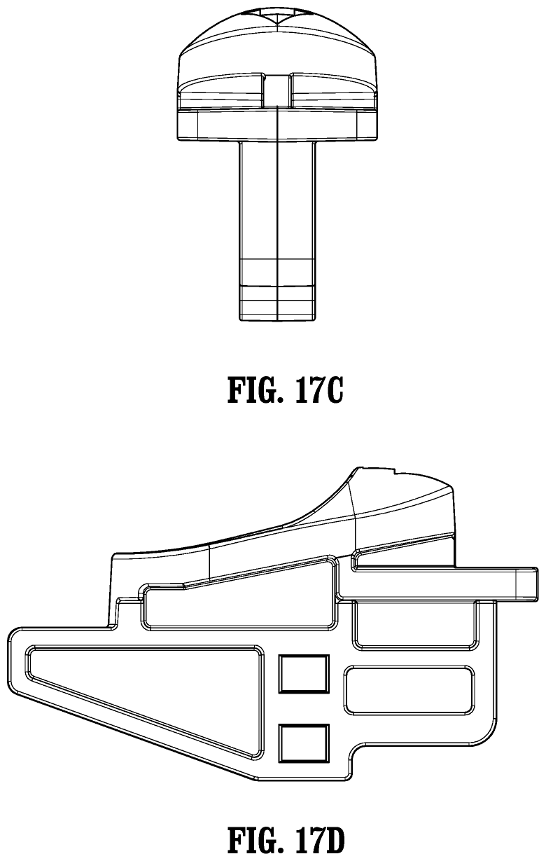

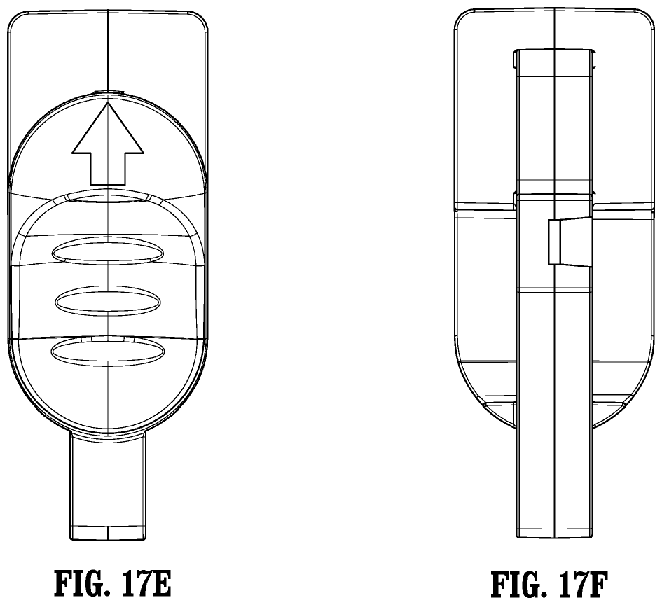

In operation, with reference to FIGS. 10, 11, 15A-15D, and 16, to detach a spent surgical loading unit 300 from the adapter assembly 400 and replace it with a new surgical loading unit 300, the instrument drive unit 52 along with the attached adapter assembly 400 and surgical loading unit 300 are moved proximally along the slide 43 (FIG. 9) of the surgical robotic arm 40 to withdraw the surgical loading unit 300 from a surgical site, as shown in step 500 in FIG. 16. In response to a first trigger threshold signifying that removal of a spent surgical loading unit 300 is desired, the processor 57 is configured to automatically actuate the motor 59 of the instrument drive unit 52 to drive a rotation of the drive screw 416 in a direction that drives a corresponding proximal movement of the drive nut 418 therealong, as shown in step 502 in FIG. 16. The first trigger threshold may be met when the instrument drive unit 52, along with the attached adapter assembly 400 and surgical loading unit 300, is moved to a proximal-most position (or near a proximal-most position) on the slide 43 (e.g., via a sensor or camera). In other aspects, the first trigger threshold may be met when the system 10 determines that a staple firing of the surgical loading unit 300 is complete. In response, the system 10 automatically enters a detaching condition, whereby the system 10 may automatically open the jaws 306, 308 (FIG. 1) of the surgical loading unit 300 to release tissue, and enable the adapter assembly 400 to be moved along slide 43.

In continuation of step 502, due to the elongate loading bar 410 being coupled to the drive nut 418 via the switch 404, the proximal movement of the drive nut 418 along the drive screw 416 causes the elongate loading bar 410 to move proximally to the proximal position (FIG. 10) to unlock the surgical loading unit 300 from the adapter assembly 400. In this way, a clinician is now capable of using a single hand to rotate and axially withdraw the surgical loading unit 300 from the adapter assembly 400 without having to simultaneously manually actuate the switch 404.

The clinician now has the choice between removing the surgical loading unit 300 from the adapter assembly 400 while the adapter assembly 400 remains attached to the instrument drive unit 52, or detaching the adapter assembly 400 from the instrument drive unit 52 and then removing the surgical loading unit 300 from the adapter assembly 400 while the adapter assembly 400 remains uncoupled from the instrument drive unit 52. Under the condition where the clinician removes the surgical loading unit 300 from the adapter assembly 400 while the adapter assembly 400 remains attached to the instrument drive unit 52, in step 504, a second trigger threshold is met signifying that the surgical loading unit 300 has been removed from the adapter assembly 400. For example, the system 10 detects, e.g., via a sensor, such as a hall effect sensor, or a camera, that the surgical loading unit 300 is removed from the adapter assembly 400.

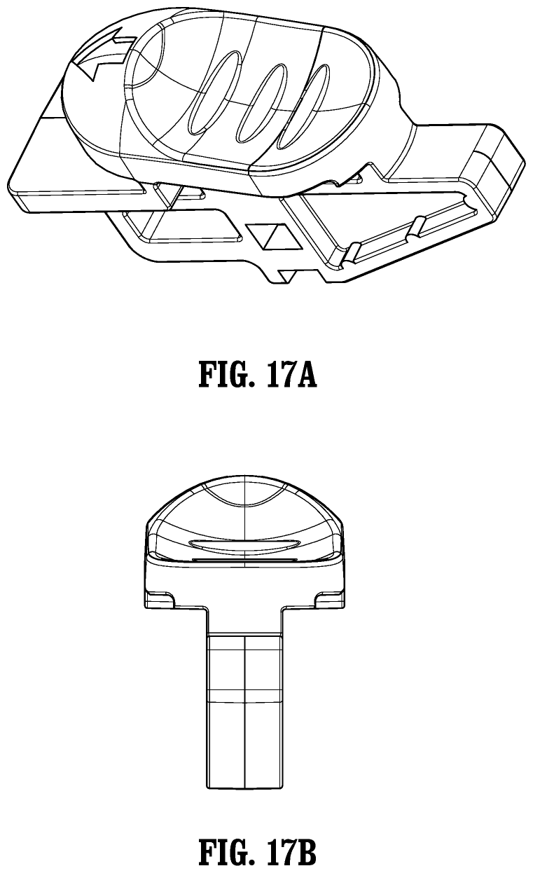

In response to step 504, in step 506, the processor 57 is configured to automatically actuate the motor 59 of the instrument drive unit 52 to drive a rotation of the drive screw 416 in a direction that drives a corresponding distal movement of the drive nut 418 therealong from a proximal position shown in FIG. 15A to a distal position shown in FIG. 15B. Due to the elongate loading bar 410 being coupled to the drive nut 418 via the switch 404, the distal movement of the drive nut 418 along the drive screw 416 causes the elongate loading bar 410 to move distally to the distal position. With the elongate loading bar 410 in the distal position, an improper insertion of a new surgical loading unit 300 into the adapter assembly 400 is prevented by the slot 414 of the elongate loading bar 410, in the manner described above.

After the elongate loading bar 410 is driven to the distal position, in step 508, the processor 57 is configured to then automatically send a command to the motor 59 of the instrument drive unit 52 to drive a proximal movement of the drive nut 418 to move the flange 420 of the drive nut 418 from the distal limit 422b of the elongate slot 422 of the switch 404 to the proximal limit 422a of the elongate slot 422, as shown in FIG. 15B. With the flange 420 of the drive nut 418 positioned at the proximal limit 422a of the elongate slot 422 of the switch 404, the switch 404 and the attached elongated loading unit 410 are free to move proximally during manual insertion of the surgical loading unit 300 into the adapter assembly 400. Without having first moved the drive nut 418 proximally relative to the switch 404, the motor 59 of the instrument drive unit 52 would resist proximal movement of the elongate loading bar 410 thereby resisting and/or preventing proximal insertion of the new surgical loading unit 300 into the adapter assembly 400.

With the elongate loading bar 410 in the distal position, in step 510, the new surgical loading unit 300 is proximally inserted into the adapter assembly 400, whereby the lug 303a of the surgical loading unit 300 engages the distal end 414 of the elongate loading bar 410 to drive the elongate loading bar 410 toward the proximal position against the resilient bias of the spring 412, as shown in FIG. 10. Upon rotating the surgical loading unit 300 out of engagement with the lug 303a of the surgical loading unit 300, as shown in FIG. 11, the spring 412 distally drives the elongate loading bar 410 into the distal position to lockingly engage the new surgical loading unit 300 with the adapter assembly 400.

In step 600, under the condition where the clinician chooses to detach the adapter assembly 400 from the instrument drive unit 52 prior to performing a surgical loading unit 300 exchange, once the adapter assembly 400 is removed from the instrument drive unit 52, any resistance to rotation of the drive screw 416 by the motor 59 of the instrument drive unit 52 is no longer present. That is, in step 602, immediately upon disengaging the adapter assembly 400 from the instrument drive unit 52, the distally-oriented force exerted by the spring 412 on the elongate loading bar 410 drives a distal movement of the elongate loading bar 410 toward the distal position. More specifically, as the elongate loading bar 410 moves distally, the drive nut 418 is moved distally therewith and along the drive screw 416, which is caused to rotate. As noted above, the multiple start thread 419 of the threaded distal end portion 416b of the drive screw 416 provides a reduced resistance to translation of the drive nut 418 along the drive screw 416 to allow for the force of only the spring 412 to drive the distal movement of the elongate loading bar 412.

In accordance with the disclosure, optionally, step 602 my further include manually moving the switch 404 proximally (or in any contemplated direction) to remove the spent surgical loading unit 300 from the adapter assembly 400.

With the elongate loading bar 410 in the distal position, due to the action of the spring 412, improper insertion of a new surgical loading unit 300 into the adapter assembly 400 is prevented by the slot 414 of the elongate loading bar 410, in the manner described above. In step 604, the new surgical loading unit 300 may be lockingly engaged to the adapter assembly 400, whereupon the adapter assembly 400, with the new surgical loading unit 300, may be re-engaged to the instrument drive unit 52.

It is contemplated that the system 10 may be configured to display on the displays 23, 32, or 34 an animation of the various states of the exchange of the spent surgical loading unit 300 with a new surgical loading unit 300. Additionally, or alternatively, the system 10 may be configured to provide an audible alert, haptic feedback, and/or a color change during each step of the exchange.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A surgical robotic system, comprising:
a surgical loading unit;
an adapter assembly configured to be operably coupled to a surgical robotic arm, the adapter assembly including:
an elongate body including a distal end portion configured to removably couple to a proximal end portion of the surgical loading unit; and
an elongate loading bar coupled to the elongate body and configured to move relative to the elongate body between a proximal position, in which the elongate loading bar is configured to allow the surgical loading unit to be removable from the elongate body, and a distal position in which the elongate loading bar is configured to secure the surgical loading unit to the elongate body;
a processor; and
a memory in communication with the processor and having instructions stored therein, the processor being configured to execute the instructions to cause the system to automatically drive a proximal movement of the elongate loading bar to the proximal position in response to a first trigger threshold.

2. The surgical robotic system according to claim 1, wherein the first trigger threshold includes the adapter assembly being moved proximally to a proximal position on the surgical robotic arm.

3. The surgical robotic system according to claim 1, wherein the adapter assembly includes:
a drive screw configured to be rotated by a motor of the surgical robotic system; and
a drive nut threadedly engaged to the drive screw and coupled to the elongate loading bar such that rotation of the drive screw translates the elongate loading bar between the proximal and distal positions via the drive nut.

4. The surgical robotic system according to claim 3, wherein the adapter assembly includes a switch coupling the drive nut to the elongate loading bar such that movement of the drive nut along the drive screw is configured to move the elongate loading bar from the distal position to the proximal position via the switch.

5. The surgical robotic system according to claim 4, further comprising a biasing member resiliently biasing the switch from a proximal position, in which the elongate loading bar is in the proximal position, toward a distal position, in which the elongate loading bar is in the distal position.

6. The surgical robotic system according to claim 5, wherein the processor is further configured to automatically cause the system to drive a distal movement of the drive nut to move the switch to the distal position thereof in response to a second trigger threshold, the second trigger threshold including the surgical loading unit being removed from the elongate body.

7. The surgical robotic system according to claim 6, wherein the drive nut includes a flange received in an elongate slot defined in the switch, wherein the processor, after causing the system to distally move the drive nut to distally move the switch to the distal position, is further configured to automatically drive a proximal movement of the flange of the drive nut within the elongate slot of the switch to position the flange at a proximal limit of the elongate slot.

8. The surgical robotic system according to claim 5, wherein the drive screw has a multiple start thread to allow for rotation of the drive screw during distal translation of the drive nut along the drive screw due to a resilient bias imparted on the drive nut by the biasing member.

9. The surgical robotic system according to claim 1, wherein the elongate loading bar has a distal end defining a slot therein configured for receipt of a proximal end portion of a component of the surgical loading unit upon an improper insertion of the surgical loading unit into the adapter assembly, whereby the engagement of the proximal end portion of the component with the slot of the elongate loading bar resists rotation of the surgical loading unit relative to the adapter assembly toward an assembled state.

10. A surgical robotic system, comprising:
an instrument drive unit having a motor;
an adapter assembly including:
a housing configured to be operably coupled to the instrument drive unit;
a manual switch slidably coupled to the housing and operably to the motor of the instrument drive unit;
an elongate body having a proximal end portion coupled to the housing, and a distal end portion configured to couple to a proximal end portion of a surgical loading unit; and
an elongate loading bar slidably coupled to the elongate body and coupled to the manual switch such that the elongate loading bar is configured to move relative to the elongate body from a distal position to a proximal position in response to proximal movement of the manual switch, the elongate loading bar being resiliently biased toward the distal position;
a processor; and
a memory in communication with the processor and having instructions stored therein, the processor being configured to execute the instructions to cause the system to:
actuate the motor of the instrument drive unit to drive a proximal movement of the elongate loading bar to the proximal position upon in response to a first trigger threshold signifying that the surgical loading unit is to be detached from the adapter assembly; and
drive a distal movement of the elongate loading bar to the distal position in response to a second trigger threshold signifying that the surgical loading unit is detached from the adapter assembly.

11. The surgical robotic system according to claim 10, wherein the surgical loading unit is configured to be rotated into a locking engagement with the adapter assembly, the elongate loading bar having a distal end defining a slot therein configured for receipt of a proximal end portion of a component of the surgical loading unit upon an improper insertion of the surgical loading unit into the adapter assembly, whereby the engagement of the proximal end portion of the component with the slot of the elongate loading bar resists rotation of the surgical loading unit relative to the adapter assembly toward the locking engagement with the adapter assembly.

12. The surgical robotic system according to claim 10, wherein the adapter assembly includes:
a drive screw operably coupled to the motor of the instrument drive unit; and
a drive nut threadedly engaged to the drive screw and coupled to the elongate loading bar via the manual switch such that rotation of the drive screw translates the elongate loading bar between the proximal and distal positions.

13. The surgical robotic system according to claim 12, wherein the drive nut includes a flange received in an elongate slot defined in the manual switch, the flange of the drive nut being configured to move between a proximal limit of the elongate slot and a distal limit of the elongate slot without moving the manual switch, wherein the processor, after causing the system to move the elongate loading bar to the distal position, is further configured to drive a proximal movement of the flange of the drive nut within the elongate slot of the switch to position the flange at the proximal limit of the elongate slot.

14. The surgical robotic system according to claim 12, wherein the adapter assembly further includes a biasing member resiliently biasing the elongate loading bar to the distal position, the drive screw having a multiple start thread to allow for rotation of the drive screw during distal translation of the drive nut along the drive screw due to the resilient bias imparted on the drive nut by the biasing member.

15. A method of exchanging a surgical loading unit in a surgical robotic system, the method comprising:
determining that a surgical loading unit is to be detached from an adapter assembly of the surgical robotic system; and
upon the system determining that the surgical loading unit is to be detached from the adapter assembly, actuating a motor of an instrument drive unit of the surgical robotic system to drive a proximal movement of an elongate loading bar of the adapter assembly to a proximal position, whereby the elongate loading bar unlocks the surgical loading unit from the adapter assembly.

16. The method according to claim 15, further comprising actuating the motor of the instrument drive unit to drive a distal movement of the elongate loading bar to a distal position upon the system determining that the surgical loading unit is detached from the adapter assembly.

17. The method according to claim 16, further comprising actuating the motor of the instrument drive unit to drive a proximal movement of a drive nut relative to a manual switch of the adapter assembly after the elongate loading bar is moved distally to the distal position.

18. The method according to claim 15, further comprising distally moving, via a biasing member of the adapter assembly, the elongate loading bar to the distal position upon detachment of the adapter assembly from the instrument drive unit.

19. The method according to claim 18, wherein the biasing member moves the elongate loading bar distally against a resistive axial force of a drive nut of the adapter assembly that intercouples a drive screw of the adapter assembly to the elongate loading bar.

20. The method according to claim 15, wherein determining that the surgical loading unit is to be detached includes determining that the adapter assembly is moved proximally to a proximal position on a surgical robotic arm of the surgical robotic system.
